(19) United States
(12) Patent Application Publication
(10) Pub. No.: US 2024/0156449 A1
(43) Pub. Date: May 16, 2024

(54) QUICK STITCH

(71) Applicant: Pedro Mendoza, Riverside, CA (US)

(72) Inventor: Pedro Mendoza, Riverside, CA (US)

(21) Appl. No.: 17/985,725

(22) Filed: Nov. 11, 2022

(51) Int. Cl. A61B 17/04

(52) U.S. Cl. CPC A61B 17/0469; A61B 17/0467; A61B 17/0482

(57) ABSTRACT

A device and method for repairing an affected area is presented. The device has a casing which has a top, a first side, a second side, a third side, a fourth side, a bottom, a driver mechanism means and a needle. The top side of the casing has a first opening substantially near the center of the top side of the casing and configured to accommodate the driver mechanism means. The driver mechanism means connects the skin of the affected area, delivers suture thread to the needle and activate the needle to insert the suture thread onto each side of the connected skin. The driver mechanism cuts the suture thread after the suture thread has been inserted on both side of the connected skin of the wound. The stitching device may have a round corner to accommodate and provide comfort for a person's hand.

### FIELD OF THE INVENTION

The present invention relates generally to the closure of wounds in body tissue, and more particularly to an automated suturing of wounds.

### BACKGROUND

Transluminal medical procedures have seen a marked rise in popularity during the past decade due to the much-reduced surgical damage to healthy tissue, recovery time, and ultimate cost to the patient associated with these procedures. Such procedures typically require an incision formed in a body lumen and the overlying tissue through which catheters, guide wires, laparoscopes, endoscopes, and similar medical devices can be inserted into the patient's vascular system. The incisions are created with instruments such as trocars, and may measure from 5 to upwards of 15 mm in diameter. After the intravascular procedure is complete, the incision must be closed and sealed as quickly as possible to prevent further bleeding.

Unfortunately, the size of the incision is usually large enough to permit profuse bleeding, but too small for the convenient application of conventional wound closure techniques such as suturing. Other methods have been proposed, including the use of collagen and other types of plugs placed outside the lumen and near the wound.

Some plugs are designed to be placed directly within the lumen and secured against the inner wall of the lumen by an external suture attached to the plug. In yet other similar closure methods, a clotting agent such as a foam, gel, or powder is dispensed within the wound. These methods all tend to cause the at least partial deposition of a plug or clotting agent within the lumen, whether by design or unintentionally, and thus result in the partial occlusion of the flow of blood through the lumen near the site of the wound. This is an unfortunate side effect that can lead to complications such as thrombosis and is therefore undesirable.

Another common closure technique employs continuous pressure applied to the wound site to allow the patient's blood to clot sufficiently to seal the wound. This technique, however, is usually very time consuming for both patient and medical personnel, thus partially negating one of the main advantages of transluminal procedures. In addition, a hematoma may occur if the patient moves while the pressure is being applied and therefore additional rest and observation in the hospital is required, further driving up the cost and time associated with the procedure.

Sutures remain the preferred method of sealing wounds, but the small size of the typical wound formed during a transluminal procedure renders the application of sutures a delicate and difficult task. Traditionally the surgeon would introduce a suture needle through the tissue tract and into the body lumen, manipulate the needle with a grasping instrument into position, then urge it to penetrate and pass through the lumen wall near the wound site and pull the suture material therethrough. This procedure involves difficult manipulations and maneuvers in a small space and through a small opening, and is therefore time consuming and burdensome.

Due to the ever-increasing number of transluminal procedures performed each year, a great deal of effort has been devoted to the development of quicker, safer methods for post-operative suturing of endoscopic wounds. Practically all viable methods involve the use of a specially developed device for placing sutures in the lumen and through the body tissue. Some devices employ needles to carry opposite ends of a suture through the tissue surrounding the wound, and then means for retrieving the ends of the suture from within the body lumen and out through the wound. Such means may include an expandable mesh, a flexible membrane capturing anchors attached to the ends of the suture, and an expandable bow assembly.

A drawback to the method employed by these devices is that the suture ends pass from within the lumen and out through the wound opening, and are thereby trapped between the tissue walls surrounding the wound when the suture is tensioned to close the opening. This suture configuration prevents the tissue walls from fully contacting each other and thus may require an extended period of time to heal.

In addition, this configuration also requires that the suture knot tied by the surgeon must lie within the lumen, and therefore in most cases the surgeon cannot tie a sufficiently tight knot to prevent any further bleeding and to ensure minimal scarring of the tissue upon healing. Finally, most such devices require the surgeon to blindly capture the ends of the suture within the lumen while blood courses through the lumen with the patient's heart beat, making exact placement and capture of the suture ends difficult and imprecise.

Other suturing devices employ tubes carrying needles mounted on extendible platforms that advance out of the distal end of the tube once the distal end has been positioned within the lumen. The needles may be straight or curved, and carry sutures so that when the device is retracted from the lumen the needles pierce the side walls of the wound and thereby carry the sutures through the surrounding tissue.

A relatively similar device has a distal end with a suture equipped with ferules at either end placed therein. The distal end is positioned within the lumen and beneath the side walls of the wound, and sharp needles are driven through the side wall tissue to engage the ferules and pull the suture out. These devices are preferable because they create a cleaner, tighter seal of the wound, and are somewhat easier to operate because the suture ends do not have to be captured blindly within the lumen.

Unfortunately, however, such devices are relatively complex and employ a significant number of moving parts. These devices are therefore relatively costly to manufacture and are also more prone to mechanical failure. In addition, such devices cannot be easily manufactured with very small diameters because of the complex mechanisms that must fit entirely within the device, especially when the needles are curved, and thus some of the devices may not be suited to suturing small diameter wounds.

The need continues to exist for a suturing method that will cleanly and tightly suture small as well as larger diameter wounds with an uncomplicated, dependable, inexpensive apparatus that is fail proof and simple to use.

### BRIEF DESCRIPTION OF THE DRAWINGS

FIG. 1 is an illustrated view of an exemplary stitching device.

FIG. 2 is an illustrated of the stitching device of FIG. 1 suturing a wound.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

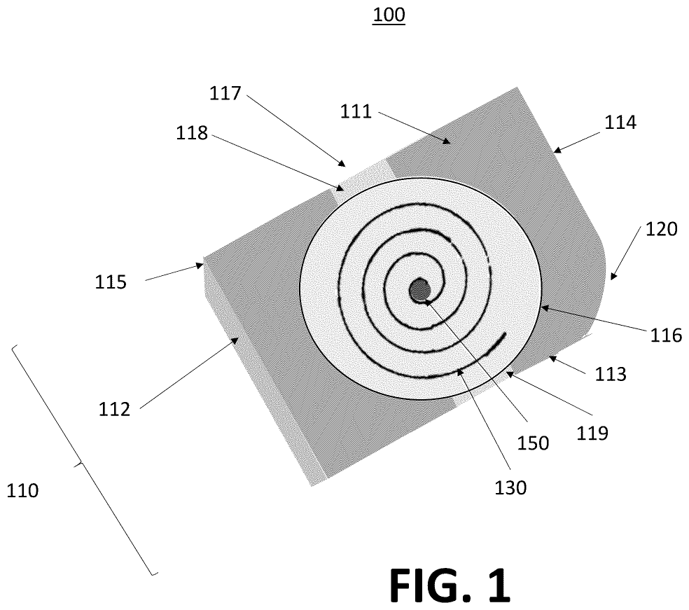

Referring to FIG. 1, a stitching device 100 for suturing wounds or cuts on a person is presented. The stitching device 100 is useful for being moved along an affected area, closing skin of the affected area, and suturing the skin of the affected area to close the affected area. The suturing device 100 reduces the time and effort to suture a wound or the affected area by moving along the wound or the affected area and suturing as the skin is joined together closing the wound or affected area.

The stitching device 100 has a casing 110, a drive mechanism 130, and a needle 150. The casing 110 is useful for providing sutures and the needle 150 in a convenient container to easily control the sutures and the needle 150. The case 110 is preferably made of a medical grade plastic material, but may be any material that is medical grade. The medical grade plastic material is strong and light weight, allowing for the stitching device 100 to be easily managed by a person as well as easy to transport.

The casing 110 has a top 111, a first side 112, a second side 113, a third side 114 and a bottom 115. The top 111 is a substantially flat surface. The top 111 has a first opening 116 substantially in the center 117 of the top 111 to couple the suture holder 130 to the casing 100. The top 111 is second opening 118 of the second side 113 and a third opening 119 of the fourth side 115 allowing easy access to the suture holder 130.

Optionally or additionally, the casing 110 may have a round corner 120. The round corner 120 is configured to be coupled to the second side 113 and coupled to the third side 113. The round corner 120 is useful for having the stitching tool 100 fit more easily and comfortably in the person's hand. Further, the rounded corner 118 may provide a more stable stitching device 100 when in use and thus a safer, easier and secure use of the stitching device 100.

The drive mechanism 130 is useful for activating the use of sutures (not shown) and the activity of the needle 150 to secure the wound or affected area. The sutures are useful when closing the wound or the affected area to be used to secure the closing of the wound or affected area until the wound or the affected area has sufficiently healed such that the sutures are no longer necessary to secure the closure of the wound or the affected area.

The suture thread may be placed into the stitching device 100 using access from the second opening 118 of the second side 113 and the third opening 119 of the fourth side 115. The suture thread may alternatively be placed in the stitching device 100 using the first opening 116 in substantially the center 117 of the top 111 of the casing 110 of the stitching device. The suture thread may be any type of suture such as nylon, polypropylene, silk, polyester, etc.

The needle 150 of the stitching device 100 is useful for applying and/or securing the suture thread to the wound or the affected area. The needle 150 may be any type of needle such as trigger point, circle taper, small have circle, cutting needles, etc.

The stitching device 100 is actuated by squeezing on the first side 112 and the third side 114. The squeezing is generally by thumb and index finger of the person but may be any fingers or other part of a hand of the person. The squeezing of the first side 112 and the third side 114 activates a drive mechanism means 130 contained within the stitching device 100.

While being squeezed, the stitching device 100 is moved along the wound or the affected area, the drive mechanism means 130 pulls the skin of the wound or the affected area tightly together and accesses the suture thread. The drive mechanism means 130 then drives the needle 150 through both side of the wound or the affected area and ties the suture thread such that skin of the wound or the affected area is secured together to start the healing process.

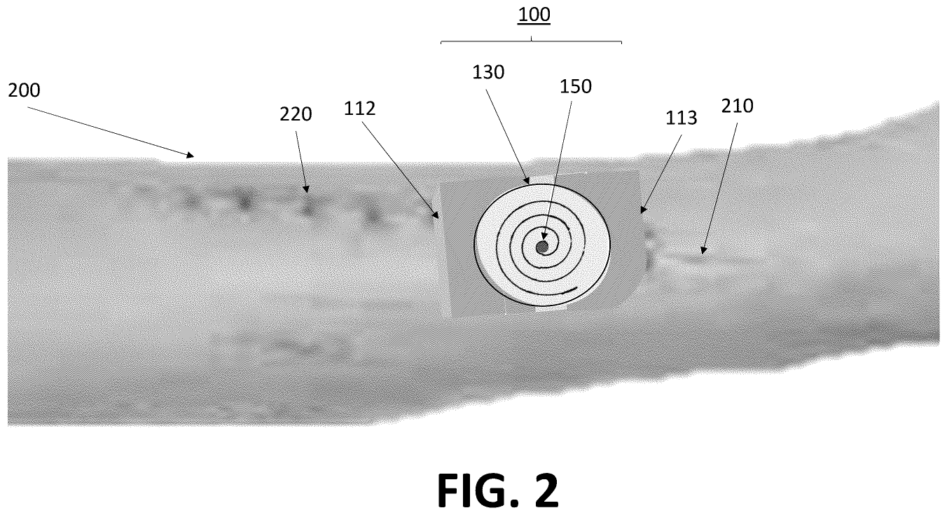

Moving now to FIG. 2, the stitching device 100 of FIG. 1 suturing a wound is illustrated.

A limb 200 has a wound 210. The wound 210 may be any type of wound but for this example the wound 210 is a long open area along the limb 200.

The stitching device 100 has been placed onto the wound 210 of the limb 200. The stitching device 100 is being squeezed at the first side 112 and the third side 114. Once placed on the wound 210, the stitching device 200 has been moved along the wound 210 while being sutured. The area of the wound 210 on the limb 200 has been closed by the stitching device 100 and sutures 220 are shown after the drive mechanism means 130 has been activated by the squeezing of the first side 112 and the third side 114 of the stitching device 100 thus accessing the suture thread in the stitching device 100 and actuation of the needle 150.

While being squeezed, the stitching device 100 is moved further along the wound or the affected area until the entire wound has been closed and secured by the sutures 220. Therefore, the drive mechanism means 130 pulls the skin of the wound or the affected area tightly together and accesses the suture thread in the stitching device 100. The drive mechanism means 130 then drives the needle 150 through both side of the wound or the affected area and ties the sutures 220 such that skin of the wound or the affected area is secured together to start the healing process.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A stitching device for repairing an affected area, comprising:
a casing, the casing having a top, a first side, a second side, a third side, a fourth side, a bottom, a driver mechanism means and a needle;
the top side of the casing having a first opening substantially near the center of the top side of the casing, the first opening configured to accommodate the driver mechanism means;
the driver mechanism means configured to connect skin of the affected area, deliver a suture thread to the needle and activate the needle to insert the suture thread onto each side of the connected skin; and
the driver mechanism cutting the suture thread after the suture thread being coupled to the connected skin of the affected area.

2. The stitching device of claim 1, wherein the stitching device being made of a medical grade plastic.

3. The stitching device of claim 1, wherein the needle being a cutting needle.

4. The stitching device of claim 1, wherein the needle being a trigger point needle.

5. The stitching device of claim 1, further comprising:
a rounded corner, the rounded corner being configured to be coupled the second side of the casing and configured to be coupled to the third side of the casing.

6. The stitching device of claim 1, wherein the suture thread being a nylon material.

7. The stitching device of claim 1, wherein the needle being configured to extend through the bottom of the casing.

8. A method of stitching a wound, the method comprising:
inserting suture thread into a stitching device;
inserting a needle into the stitching device;
placing the stitching device onto a wound;
activating a driver mechanism means configured to access the suture thread and activating movement of the needle; and
after the suture thread has been coupled to the wound, cutting the suture thread.

9. The method of claim 8, wherein the thread being a nylon material.

10. The method of claim 8, wherein the needle being a cutting needle.

11. The method of stitching a wound, further comprising:
moving the stitching device on the wound; and
actuating the driver mechanism means to apply the suture thread to the connected skin of the wound.

12. The method of claim 8, wherein the stitching device having at least one rounded corner.
